(19) United States
(12) Patent Application Publication
(10) Pub. No.: US 2021/0145295 A1
(43) Pub. Date: May 20, 2021

(54) IMAGE PROCESSING APPARATUS AND METHOD AND IMAGE PROCESSING SYSTEM

(71) Applicant: SONY CORPORATION, TOKYO (JP)

(72) Inventors: GORO FUJITA, KANAGAWA (JP); TETSURO KUWAYAMA, TOKYO (JP); HIROSHI ICHIKI, KANAGAWA (JP)

(21) Appl. No.: 16/611,545

(22) PCT Filed: May 2, 2018
(86) PCT No.: PCT/JP2018/017483

§ 371 Date: Nov. 7, 2019

(30) Foreign Application Priority Data
May 16, 2017 (JP) 2017-097164

(51) Int. Cl. A61B 5/026; A61B 5/00; G06T 7/00

(52) U.S. Cl. CPC A61B 5/0261; A61B 5/0066; G06T 2207/10048; G06T 2207/30104; G06T 7/0012

(57) ABSTRACT

The present disclosure relates to an image processing apparatus and method and an image processing system that enable highly accurate observation. An intra-frame operation unit performs, as online processing, image processing of speckles generated by irradiation with laser light on a captured image in accordance with a relationship between an image output frame rate and a sampling rate. A high-precision operation unit performs, as offline processing, the image processing of speckles on the captured image in accordance with the relationship between an image output frame rate and a sampling rate. The present disclosure may be applied to an image processing system including, for example, a speckle imaging apparatus.

### TECHNICAL FIELD

The present disclosure relates to an image processing apparatus and method and an image processing system, and more particularly to an image processing apparatus and method and an image processing system that enable highly accurate observation.

### BACKGROUND ART

There is a technique of observing blood flow through a camera optical system or the like by using speckles generated by irradiation with laser light. Patent Document 1 describes methods for contrast calculation processing of speckles as follows. A method using time-dependent intensity changes is excellent in spatial resolution. A method for measuring dispersion of a spatial area is excellent in time response. Patent Document 1 describes selection from among the methods depending on the purpose.

### CITATION LIST

### Patent Document

Patent Document 1: PCT Japanese Translation Patent Publication No. 2016-533814

### SUMMARY OF THE INVENTION

### Problems to be Solved by the Invention

However, Patent Document 1 does not describe the use of different contrast calculation processing of speckles in online and offline states.

The present disclosure has been made in view of such a situation so as to enable highly accurate observation.

### Solutions to Problems

An image processing apparatus according to an aspect of the present technology includes a control unit that controls whether to perform image processing of speckles as online processing or offline processing on a captured image in accordance with a relationship between an image output frame rate and a sampling rate, the speckles being generated by irradiation with laser light.

In a case where the captured image is acquired at a sampling rate equal to an image output frame rate, the control unit can perform, as the online processing, image processing of speckles to be completed within a frame, and perform, as the offline processing, image processing of speckles that requires inter-frame processing.

In a case where the captured image is acquired at a sampling rate equal to an image output frame rate, the control unit can perform, as the online processing, image processing of speckles that requires inter-frame processing, by replacing a corresponding frame with a previous frame depending on information of a plurality of frames preceding the corresponding frame.

In a case where the captured image is acquired at a sampling rate higher than an image output frame rate, the control unit can perform: as the online processing, image processing between a plurality of sample frame images in the output frame within the output frame rate, in addition to image processing of speckles to be completed within a sampling frame; and as the offline processing, arithmetic processing that does not fit within the output frame rate on the captured image stored in a memory.

The control unit can perform writing of the captured image to the memory and the arithmetic processing as the offline processing in parallel with the image processing of speckles as the online processing.

The control unit can perform writing of the captured image to the memory and the arithmetic processing as the offline processing after a certain period of time after the image processing of speckles as the online processing.

The inter-frame processing is processing for excluding a frame that reduces a speckle contrast of an entire image and outputting an optimum speckle contrast by complementing the excluded frame from preceding and following frames or by averaging other images in an output frame.

The inter-frame processing is processing in which: a plurality of exposure times is set for a sample frame in the output frame rate; a flow velocity is calculated from a contrast value for each exposure time on the basis of a previously set relational expression of a flow velocity and a contrast value for each exposure time; and a most probable flow velocity is calculated and reflected in an image.

The inter-frame processing is processing for detecting a size of a fluid part on the basis of a different captured image and optimizing a calculation cell size so as to achieve a resolution corresponding to the detected size.

The inter-frame processing is processing including laser speckle perfusion imaging (LSPI), laser speckle flowgraphy (LSFG), or frequency domain laser speckle imaging (FDLSI) which is a calculation method using information in a time direction of speckles.

The image processing apparatus can further include a switching unit that causes a display image to switch between a speckle image subjected to image processing as the online processing and a speckle image subjected to image processing as the offline processing.

An image processing method according to an aspect of the present technology, to be performed by an image processing apparatus, includes: controlling whether to perform image processing of speckles as online processing or offline processing on a captured image in accordance with a relationship between an image output frame rate and a sampling rate, the speckles being generated by irradiation with laser light.

An image processing system according to another aspect of the present technology includes: a light source that irradiates a surface of an object with laser light; and an image processing apparatus including: a control unit that controls whether to perform image processing of speckles as online processing or offline processing on a captured image in accordance with a relationship between an image output frame rate and a sampling rate, the speckles being generated by irradiation with the laser light from the light source.

In an aspect of the present technology, whether to perform image processing of speckles as online processing or offline processing on a captured image is controlled in accordance with a relationship between an image output frame rate and a sampling rate, the speckles being generated by irradiation with laser light.

In another aspect of the present technology, a light source irradiates a surface of an object with laser light. Then, whether to perform image processing of speckles as online processing or offline processing on a captured image is controlled in accordance with a relationship between an image output frame rate and a sampling rate, the speckles being generated by irradiation with the laser light from the light source.

### Effects of the Invention

According to the present technology, an image can be processed. In particular, highly accurate observation can be performed.

### BRIEF DESCRIPTION OF DRAWINGS

FIG. 1 is a diagram describing the principle of speckle imaging.

FIG. 2 is a diagram describing the principle of speckle imaging.

FIG. 3 is a diagram describing the principle of speckle imaging.

FIG. 4 is a block diagram showing a basic configuration example of an image processing system to which the present technology has been applied.

FIG. 5 is a diagram showing an example of a luminance image of a two-dimensional image.

FIG. 6 is a diagram showing an example of a speckle contrast image.

FIG. 7 is a diagram describing a process for distinguishability of speckles.

FIG. 8 is a diagram showing an example of a user IF.

FIG. 9 is a diagram describing the effect of speckle vibration.

FIG. 10 is a diagram describing determination of the flow velocity of a speckle image.

FIG. 11 is a diagram showing a graph concerning determination of the flow velocity of a speckle image.

FIG. 12 is a flowchart describing a flow velocity determination process for a speckle image.

FIG. 13 is a block diagram showing a first configuration example of the image processing system to which the present technology has been applied.

FIG. 14 is a block diagram showing a second configuration example of the image processing system to which the present technology has been applied.

FIG. 15 is a diagram describing an operation example of a speckle imaging apparatus shown in FIG. 13.

FIG. 16 is a diagram describing an operation example in which a process for vibration is performed online.

FIG. 17 is a block diagram showing a third configuration example of the image processing system to which the present technology has been applied.

FIG. 18 is a diagram describing an operation example of a speckle imaging apparatus shown in FIG. 17.

FIG. 19 is a diagram describing an example of an operation in which exposure control has been added.

FIG. 20 is a diagram describing threshold processing to be added at the end of basic processing.

FIG. 21 is a diagram describing the threshold processing to be added at the end of the basic processing.

FIG. 22 is a block diagram showing a main configuration example of a computer.

### MODE FOR CARRYING OUT THE INVENTION

Modes for carrying out the present disclosure (hereinafter referred to as embodiments) will be described below. Note that description will be provided in the following order.

0. Overview

1. Embodiments

2. Computer

### 0. OVERVIEW

<Outline of Present Technology>

The present technology will be described mainly on the basis of a method that requires observation of blood flow in brain surgery. However, the clinical department is not particularly limited thereto. The present technology is intended for a technique or apparatus effective in observing the flow of bodily fluid including lymph as well as blood flow in surgery.

The use of speckle imaging for observation of blood flow in brain surgery is being studied. In a case of a cerebral aneurysm, an aneurysm is embedded in between wrinkles in a brain. Cerebral aneurysm clipping is an operation in which the wrinkles are carefully removed to allow clipping so that rupture is prevented. As a result of occluding the base of the aneurysm with a clip in the operation, it is possible to completely stop blood flow to the aneurysm. In doing so, it is necessary to confirm complete occlusion of the clipped region. Then, the use of speckle imaging can achieve the following effects: it is possible to perform the operation while observing whether blood flow has been stopped by the clipping (effect 1), and in addition, the complete occlusion can be finally confirmed to finish the clipping (effect 2).

Meanwhile, in vascular bypass surgery, a giant cerebral aneurysm refers to an aneurysm with a maximum diameter of 25 mm or more, and is mainly treated with craniotomy. In many cases, however, it is difficult to directly clip the aneurysm. In such cases, treatment is provided on the basis of a method for alternatively creating a bypass after occluding an artery at a position upstream of the aneurysm developed in the artery. It is necessary to confirm blood flow in the created bypass also in this case.

With regard to image processing for observation of blood flow in such a case as (effect 1), it is desirable to perform real-time online processing that causes no delay, so as to provide feedback on observation results on the spot. To be precise, it is desirable to perform processing within a standard refresh rate that enables a user to operate an apparatus without uncomfortable feeling.

Meanwhile, in such a case as (effect 2), work is temporarily stopped during an operation to confirm complete occlusion. Thus, real-time processing is not required. Therefore, offline processing is more suitable for more accurate flow observation. Furthermore, this image is also useful for postoperative analysis.

If it is possible to freely switch between online processing and offline processing during an operation both in (specific example 1) and (specific example 2) to be described later, an operating surgeon can sequentially obtain information suitable for the purpose. This can contribute to improvement in operation accuracy.

First, as (specific example 1), description will be provided on the basis of the following case: “an operating surgeon performs an operation while observing in the viewfinder of an operation microscope, and the same field image is displayed on a monitor at an operation site by use of a camera placed in a bifurcated optical path in the microscope”.

Indocyanine green (ICG) is often used to observe blood flow in brain surgery. In the case of ICG, in addition to an RGB camera image, a fluorescent image of an IR light camera is displayed on the monitor to observe blood flow. Furthermore, there is also a device which overlays the fluorescent image on an image of the viewfinder.

Therefore, taking the example of applying the present technology to the clipping method, offline processing is suitable in a case where work is stopped during the operation to confirm complete occlusion on the monitor at the operation site. This is because a highly accurate image reflecting flow velocity is more useful than a real-time image in such a case. Furthermore, in a case where a speckle image is overlaid in the viewfinder of a surgical field, it is preferable to display the speckle image or overlay the speckle image on an RGB image by real-time online processing that causes no delay in display.

Next, as (specific example 2), description will be provided on the basis of the following case: “an operating surgeon performs an operation while observing a surgical field monitor with a video microscope, and performs a check of an assistant or other checks during the operation on a large monitor provided along with the surgical field monitor”.

Observation of blood flow is performed while an RGB camera image and a fluorescent image of an IR light camera are displayed in parallel on the monitor provided along with the surgical field monitor at the operation site. It is also conceivable that the IR image is overlaid on an RGB monitor of the surgical field.

Therefore, with regard to the case of applying the present technology to the clipping method, offline processing is suitable in a case where work is stopped during the operation to confirm complete occlusion in the surgical field or on the monitor provided along with the surgical field monitor. This is because a highly accurate image reflecting flow velocity is required rather than a real-time image in such a case, and inter-frame processing can be easily performed offline. Furthermore, in a case where a speckle image is overlaid on the surgical field monitor during the operation, real-time online processing is preferable since real-time online processing causes no delay in display.

As described above, it can be expected that the present technology improves usefulness in medicine by allowing a surgeon to freely select an image processed in online processing or an image processed in offline processing as an image to be displayed on the surgical field monitor or the monitor at the operation site, according to surgical techniques.

<Principle of Speckle Imaging>

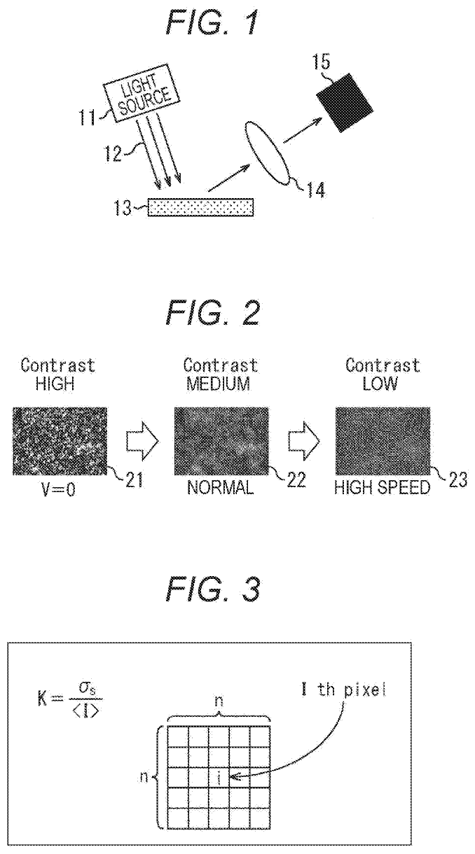

FIGS. 1 to 3 are diagrams describing the principle of speckle imaging used in the present technology.

As shown in FIG. 1, a light source 11 irradiates an object surface 13 with coherent light 12 such as laser light. The coherent light 12 is reflected from the object surface 13. The reflected light is imaged by a lens 14 to produce random interference fringes 15.

The random interference fringes (interference pattern) 15 can be observed. As shown in FIG. 2, the random interference fringes 15 have a high contrast between light and dark if the velocity V of an object is set as V=0. In addition, if the velocity is normal, the contrast is medium, and if the velocity is high, the contrast is low. In other words, the random interference fringes 15 become blurred as the velocity increases.

As described above, the contrast is low in an area where there is movement due to blood flow or the like, and the random interference pattern (referred to as a speckle pattern) is generated in an area other than the area where there is movement. Thus, the area where there is movement looks different from the other area where there is no movement. The contrast of the interference fringes 15 is referred to as speckle contrast.

FIG. 3 shows the definition of speckle contrast. The pixels of n rows and n columns form a calculation cell. The speckle contrast for the I-th pixel in the calculation cell is represented by equation (1) below.

$$\begin{matrix}[ Math . 1 ] & \\ K = \frac{{σ}_{s}}{〈 I 〉} & ( 1 )\end{matrix}$$

The standard deviation represents the spread of distribution of light and dark in a small area of an image.

<Speckle Operation Principle>

Next, the speckle operation principle will be described.

The speckle operations include a spatial contrast calculation called laserspectrumcontrustanalysis (LASCA) and a temporal contrast calculation called laserspckleimaging (LSI).

When the calculation cell has m rows and n columns, the spatial contrast calculation is represented as:

Speckle Contrast=σ(I_{m, n})/Ave(I_{m, n}).

The spatial contrast calculation is processing to be completed within a frame. Furthermore, the spatial contrast calculation is high in time-axis resolving power. Thus, when m×n is increased, the contrast increases but spatial resolving power decreases. In addition, the amount of memory is small as a calculation load. Therefore, the spatial contrast calculation is suitable for high-speed processing (online processing).

Meanwhile, when the time T is set as T=i, the temporal contrast calculation is represented as:

Speckle Contrast=σ(Iᵢ)/Ave(Iᵢ).

The temporal contrast calculation requires processing of multiple frames. Furthermore, the temporal contrast calculation is high in spatial resolving power, and is capable of velocity detection. However, the temporal contrast calculation is low in time-axis resolving power. In addition, a calculation load is large due to a plurality of frame memories. Therefore, the temporal contrast calculation is suitable for high-precision calculation (offline processing).

Moreover, several techniques combining spatial and temporal contrast calculations are being studied. For example, laser speckle perfusion imaging (LSPI) is a combination of LASCA and LSI methods, and uses temporal and spatial information. Furthermore, laser speckle flowgraphy (LSFG) is a combination of LASCA and LSI methods, and uses temporal and spatial information. Frequency domain laser speckle imaging (FDLSI) is a method for obtaining statistical characteristics of a moving object by the autocorrelation of scattered light.

Note that all of these methods require processing of a multiple frames. Therefore, these methods are suitable for high-precision calculation (offline processing).

Here, online processing is suitable for observation that is performed during an operation to be reflected in the operation on a real-time basis. The spatial contrast calculation (LASCA) which is completed within a frame is suitable for such observation.

Offline processing based on inter-frame processing is useful in a case where accuracy in flow velocity and resolution is required rather than availability on a real-time basis, such as in a case where a diagnosis of the blockage of blood flow is made while work is temporarily stopped during an operation.

A method involving the speckle operation based on different principles is also effective in performing inter-frame processing. In addition, various image processing can also be applied.

The following technique can be proposed as a technique for performing inter-frame processing online: an image is acquired at a sample rate higher than the refresh rate of a display output for observation, and inter-frame processing is completed within a display output frame.

The present technology enables inter-frame processing to be efficiently incorporated into online/offline observation so as to enhance the quality of observation during an operation, so that a user can select a proper processing method as appropriate.

Incidentally, while a display output of approximately 60 Hz is sufficient from the viewpoint of ergonomics, some of recent image sensors are applicable to a higher sampling rate (120 Hz or more). Taking into consideration future progress of sensors, it can be said that the present technology has a high feasibility.

### 1. EMBODIMENTS

<Basic Configuration Example of Image Processing System>

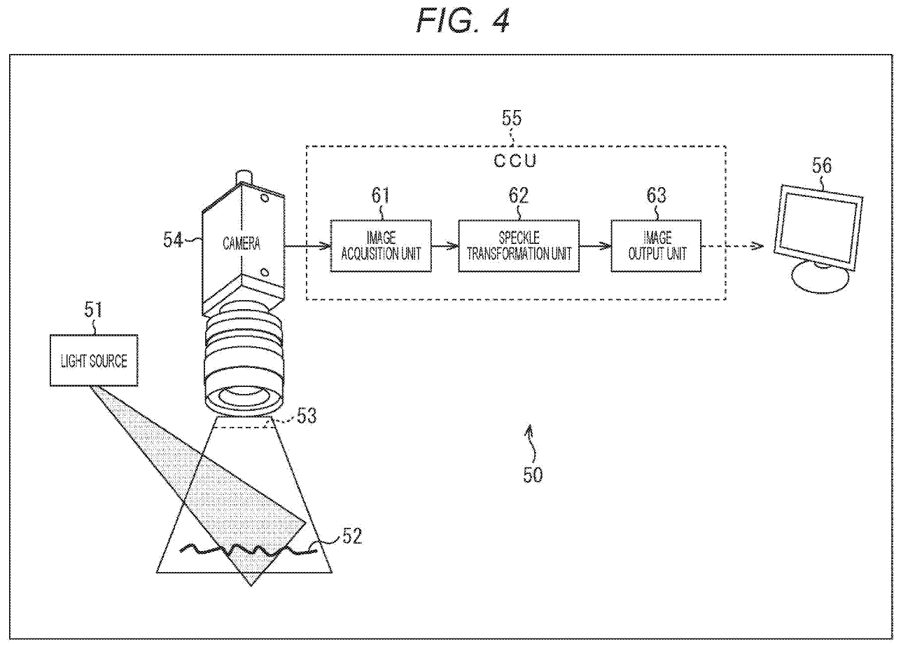

FIG. 4 is a block diagram showing a basic configuration example of an image processing system including a speckle imaging apparatus as an image processing apparatus to which the present technology has been applied.

In the example of FIG. 4, the image processing system includes a light source 51 and a speckle imaging apparatus 50 that includes a filter 53, a camera 54, a CCU 55, and a display unit 56.

The light source 51 is, for example, a narrow-band IR light source, and irradiates an object surface 52 with laser light (coherent light). Note that any light source may be used as long as the light source emits coherent light. The camera 54 includes, for example, a CMOS, a CCD, and an imager. The camera 54 captures an image of the object surface 52 via the filter 53, and supplies the resultant captured image to the CCU 55.

The CCU 55 includes an image acquisition unit 61, a speckle transformation unit 62, and an image output unit 63. The image acquisition unit 61 inputs and supplies, to the speckle transformation unit 62, an image from the camera 54. The speckle transformation unit 62 performs speckle transformation on the image input by the image acquisition unit 61, and outputs the image subjected to the speckle transformation to the image output unit 63. The image output unit 63 causes the display unit 56 to display the image subjected to the speckle transformation.

<Speckle Transformation>

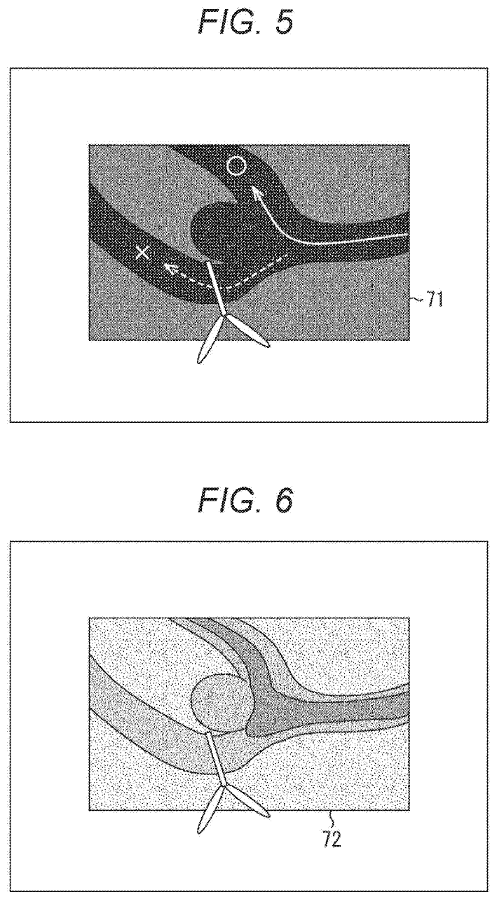

Next, speckle transformation to be performed in the speckle transformation unit 62 will be described. A two-dimensional image of, for example, w 1920×h 1080×d 12 (luminance) is captured by the camera 54 in a case of a certain HD resolution sensor. The two-dimensional image includes each luminance image 71 shown in FIG. 5. The luminance image 71 shows blood flow in a blood vessel. In the luminance image 71, blood coming from the right side is flowing upward, and downward blood flow from the right side has been stopped. Note that the white object on the blood vessel shown in the lower center is a pair of clipping forceps for holding the blood vessel.

When speckle transformation (for example, Ave (I_{0, 0}+I_{0, 1}·I_{3, 2})→Sqrt(Σ[(Im, n)−Ave]\hat{ }2)→σ/AVE) is performed on a two-dimensional image, a speckle contrast image 72 of 1920−(m−1)/2×1080−(n−1)/2 is obtained as a result of the speckle transformation in a case of HD resolution.

Next, the present technology for enhancing the quality of observation of speckles will be described on the basis of the following three items. Of these three items, intra-frame processing is preferable in terms of (1) and (3), and inter-frame processing is preferable in terms of (2).

(1) Distinguishability of Speckles

(2) Effect of Speckle Vibration

(3) Determination of Flow Velocity of Speckle Image

<Regarding Distinguishability of Speckles>

First, the distinguishability of speckles will be described.

While a fluid being observed is close to white noise, the speckle contrast of a stationary part of the background is large. Therefore, in an image transformed to luminance in accordance with the definition of speckles, the background is bright and glare remains as shown in FIG. 6. In addition, the fluid (blood flow area) is dark and not highlighted.

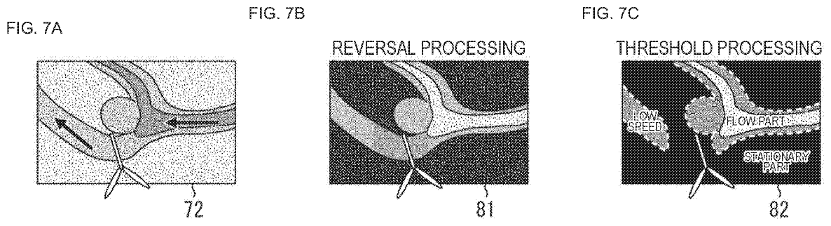

In view of this, the present technology causes luminance reversal processing to be performed on the speckle contrast image 72 as shown in A of FIG. 7, so that the speckle contrast image 72 is displayed as a highlighted image (monochrome image) so as to make the blood flow area distinguishable. An image 81 shown in B of FIG. 7 is a highlighted image (monochrome image) that has been subjected to the reversal processing. Furthermore, a highlighted image (hue image) may be displayed in addition to performing the reversal processing.

If, after such an image is displayed, threshold processing is additionally performed such that the background part is masked by the threshold processing, the blood flow area can be easily observed as shown in an image 82 in C of FIG. 7. The image 82 is an image obtained as a result of threshold processing of the highlighted image (monochrome image) that has been subjected to the reversal processing.

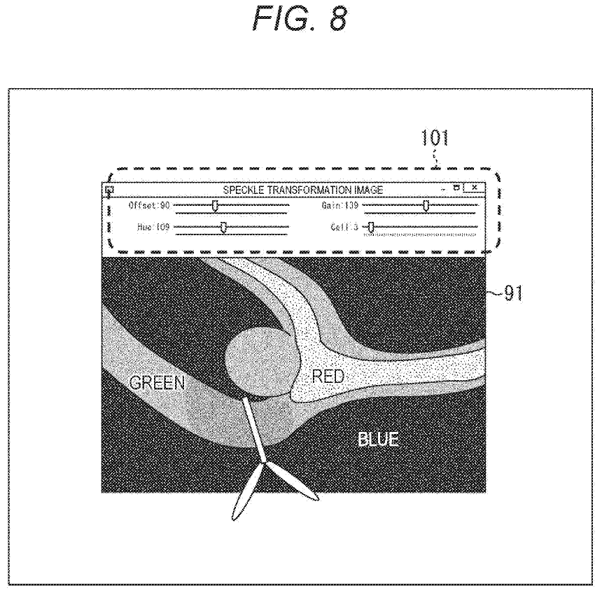

Note that the offset, gain, and threshold (hue and cell (size)) of control elements of the processing described above with reference to FIG. 7 may be, for example, changed online by the user from a user interface (IF) 101 displayed on the display unit together with an image 91 as shown in FIG. 8, or may be optimized from the image. In such a case, the cell size of speckle transformation may be determined from the size of a fluid part for which a threshold has been detected. The image 91 in FIG. 8 is a highlighted image (hue image) that has been subjected to the reversal processing. For example, a low-contrast portion of the blood flow area is shown in red, and the stationary part is shown in blue. Note that it is also possible to additionally perform threshold processing on the image 91 such that the background part is masked by the threshold processing, after displaying the image 91.

<Regarding Speckle Vibration>

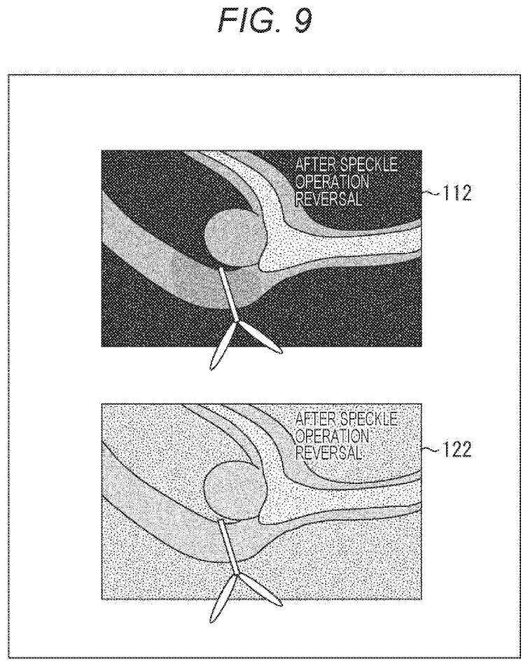

Next, the effect of speckle vibration will be described with reference to FIG. 9. When an object being observed or an imaging system vibrates, a relative velocity of the object occurs, so that the contrast of speckles decreases.

As an example, FIG. 9 shows a speckle operation reversed image 112 to be seen in the absence of speckle vibration. Furthermore, FIG. 9 also shows a speckle operation reversed image 122 to be seen in the presence of speckle vibration.

As shown in the speckle operation reversed image 112 and the speckle operation reversed image 122 described above, the object is moved due to the effect of vibration of the pair of clipping forceps for holding the blood vessel. In addition, the contrast of a part other than the blood flow also decreases. Therefore, it is difficult to distinguish the blood flow area in the presence of vibration. The effect of movement is caused also in pixel units. Thus, it can be said that speckles are highly sensitive to vibration. Meanwhile, it is difficult to distinguish changes in pixel units in an IR image or an RGB image before transformation.

Therefore, the overall luminance of each frame is calculated in the present technology to exclude a frame having a significantly higher overall luminance than the preceding and following frames. Then, the excluded frame is complemented from, for example, the preceding and following frames after the processing. Here, amplitude is reversed in speckle contrast. Therefore, contrast decreases and luminance increases due to the effect of vibration.

For example, speckle transformation is performed on input images 131-0 to 131-4 of t0 to t4 to generate transformed images 132-0 to 132-4. The pixel luminance averages of the transformed images 132-0 to 132-4 are 27.1, 23.4, 39.1, 29.9, and 30.7, respectively. The ratios of these averages to the average of five frames, that is, the transformed images 132-0 to 132-4 are 0.90, 0.78, 1.30, 0.99, and 1.02, respectively. Consequently, it is determined that the luminance of the transformed image 132-2 is significantly high. Thus, the transformed image 132-2 is excluded and then complemented from the preceding and following frames. In other words, processed images 133-0, 133-1, 133-3, and 133-4 correspond to the transformed images 132-0, 132-1, 132-3, and 132-4, respectively. Meanwhile, a processed image 133-2 has been generated while being complemented from the processed images 133-1 and 133-4. Note that the processed image 133-2 need not be a complemented image, but may be an image generated as a result of averaging a plurality of images.

<Determination of Flow Velocity of Speckle Image>

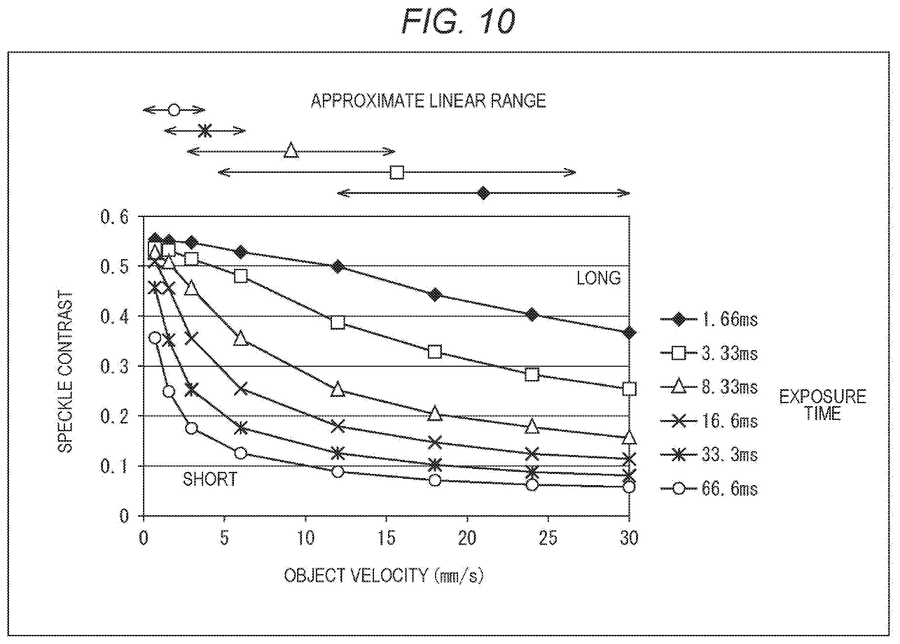

Moreover, determination of the flow velocity of a speckle image will be described with reference to FIG. 10.

It is possible to obtain an image in which the velocity of blood flow has been reflected by speckle contrast (hereinafter, also simply referred to as contrast). The graph of FIG. 10 shows results of actually measuring speckle contrast by use of a scatterer while the velocity (mm/s) of the movement of the scatterer and exposure time are changed. It can be seen from the graph of FIG. 10 that an area where a linear relationship between velocity and contrast is found or an area where detection sensitivity (gradient) is high differs according to exposure conditions.

When three different values of the exposure time T are given to the same observation pixel, the speckle contrast C is obtained for each exposure time. If the relationship (CV curve) between flow velocity and contrast for each exposure time is known in advance, the predicted flow velocity V is obtained for each exposure time.

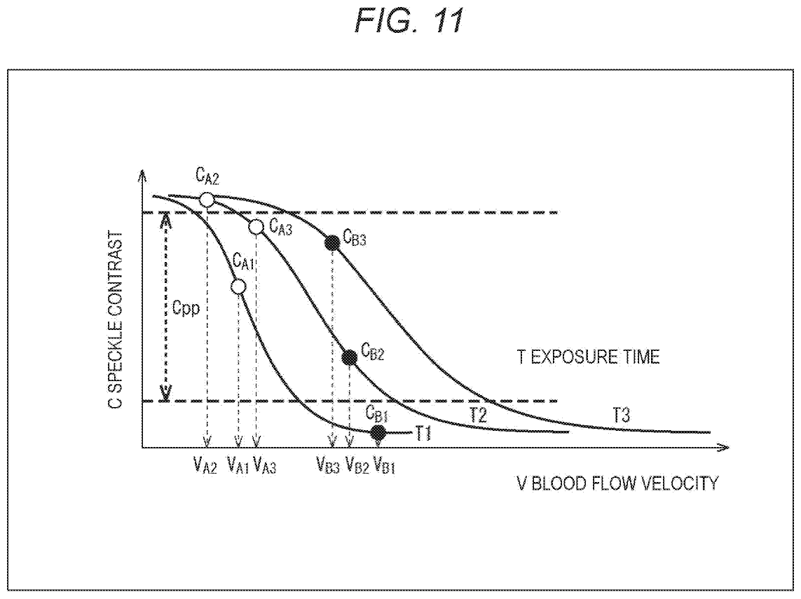

As an example, FIG. 11 shows a graph where the following values are obtained: exposure times T1, T2, and T3 are given to a pixel of A to obtain contrasts C_{A1}, C_{A2}, and C_{A3} and blood flow velocities V_{A1}, V_{A2}, and V_{A3}; and the exposure times T1, T2, and T3 are given to a pixel of B to obtain contrasts C_{B1}, C_{B2}, and C_{B3} and blood flow velocities V_{B1}, V_{B2}, and V_{B3}. It can be seen in the graph of FIG. 11 that the contrasts C_{A2} and C_{B1} are out of a measurable range Cpp.

The most probable flow velocity is calculated on the basis of the obtained three values of velocity, as follows. That is, in a case of a single exposure condition, velocity can be accurately detected within a limited linear range, while it is possible to obtain information with higher accuracy.

For example, a contrast value for each exposure time is excluded if the contrast value is out of a measurable range. Furthermore, the average of median points of speckle contrast/velocity sensitivity is taken on the basis of, for example, contrast values for respective exposure times. Moreover, different CV curves are used for calculation in the fluid part and the stationary part. Alternatively, there is used a calculation method such as a method in which the stationary part is excluded from calculation.

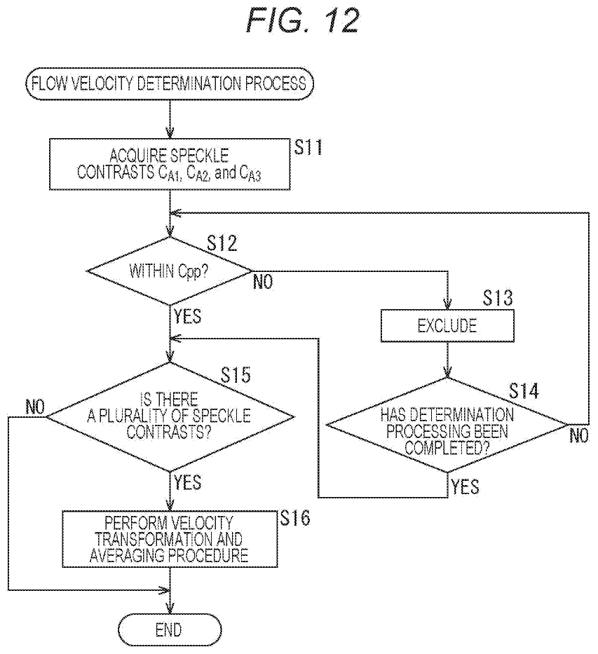

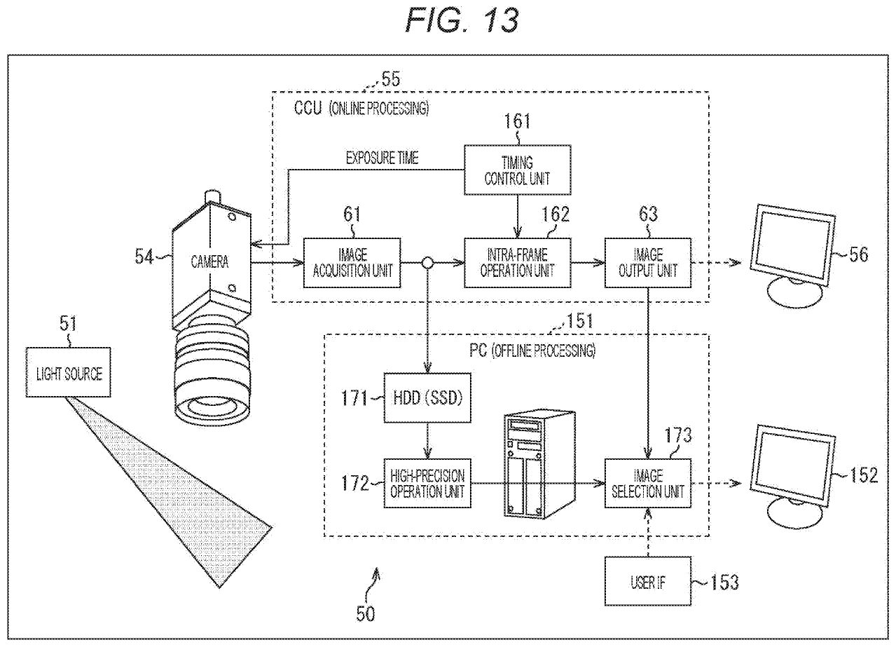

Specifically, taking the graph of FIG. 11 as an example, a flow velocity determination process is performed as shown in FIG. 12. The flow velocity determination process shown in FIG. 12 will be described by use of, for example, the speckle transformation unit 62 shown in FIG. 4. However, in fact, the flow velocity determination process is a process to be performed by, for example, an intra-frame operation unit 162 shown in FIG. 13 to be described later.

In step S11, the speckle transformation unit 62 acquires the speckle contrasts C_{A1}, C_{A2}, and C_{A3}. In step S12, the speckle transformation unit 62 sequentially determines whether or not the speckle contrasts C_{A1}, C_{A2}, and C_{A3} are within the measurable range Cpp. In a case where it is determined in step S12 that a speckle contrast is not within the measurable range Cpp, the process proceeds to step S13.

In step S13, the speckle transformation unit 62 excludes the contrast that is out of the measurable range Cpp. In step S14, the speckle transformation unit 62 determines whether or not all the Cpp determinations of the speckle contrasts C_{A1}, C_{A2}, and C_{A3} have been completed. In a case where it is determined in step S14 that a Cpp determination has not yet been completed, the process proceeds to step S12. In a case where it is determined in step S14 that all the Cpp determination processing has been completed, the process proceeds to step S15.

In a case where it is determined in step S12 that at least one of the speckle contrasts C_{A1}, C_{A2}, or C_{A3} is within the measurable range Cpp, the process proceeds to step S15.

In step S15, the speckle transformation unit 62 determines whether or not there is a plurality of contrasts within the measurable range Cpp. In a case where it is determined in step S15 that a plurality of contrasts is within the measurable range Cpp, the process proceeds to step S16. In step S16, the speckle transformation unit 62 performs an averaging procedure of T1, T2 and T3 with respect to the contrast of the measurable range Cpp from the contrasts C_{A1}, C_{A2}, and C_{A3}. The speckle transformation unit 62 treats the average value as the most probable flow velocity, and ends the flow velocity determination process.

In a case where it is determined in step S15 that there is not a plurality of contrasts within the measurable range Cpp, that is, there is just a single contrast within the measurable range Cpp, the speckle transformation unit 62 calculates a flow velocity from the contrast within the measurable range Cpp to treat the flow velocity as the most probable flow velocity, and ends the flow velocity determination process.

Specifically described below is the image processing system including the speckle imaging apparatus that performs the process for distinguishability of speckles, the process for speckle vibration, and the flow velocity determination process for a speckle image, described above with reference to FIGS. 7 to 12.

<First Configuration Example of Image Processing System of Present Technology>

FIG. 13 is a block diagram showing a first configuration example of the image processing system including the speckle imaging apparatus as an image processing apparatus to which the present technology has been applied. Note that the object surface 52 and the filter 53 have been omitted, and are not shown in the example of FIG. 13.

The image processing system shown in FIG. 13 includes the light source 51 described above with reference to FIG. 4 and the speckle imaging apparatus 50. The speckle imaging apparatus 50 includes a personal computer (PC) 151, a display unit 152, and a user IF 153 in addition to the camera 54, the CCU 55, and the display unit 56 described above with reference to FIG. 4.

Note that the speckle imaging apparatus 50 to be described below is an apparatus that performs, as online processing, image processing of speckles generated by irradiation with laser light on a captured image or performs, as offline processing, the image processing of speckles on the captured image, in accordance with a relationship between an image output frame rate and a sampling rate. Among others, the speckle imaging apparatus 50 shown in FIG. 13 is an apparatus that acquires a camera image at a sampling rate equal to an image output frame rate.

In the example of FIG. 13, the CCU 55 includes the image acquisition unit 61 and the image output unit 63 in common with the example of FIG. 4. The CCU 55 shown in FIG. 13 differs from that in the example of FIG. 4 in that a timing control unit 161 has been added and the speckle transformation unit 62 has been replaced with the intra-frame operation unit 162. The CCU 55 performs, as online processing, image processing of speckles on a captured image in accordance with a relationship between an image output frame rate and a sampling rate (a sampling rate equal to an image output frame rate in the case of FIG. 13).

In other words, the image acquisition unit 61 supplies an image from the camera 54 to the intra-frame operation unit 162 and an HDD 171 of the PC 151 in the example of FIG. 13. The timing control unit 161 controls the exposure time of the camera 54. The intra-frame operation unit 162 performs an intra-frame operation to be completed within a frame, as part of speckle transformation processing. The image output unit 63 causes the display unit 56 to display an image subjected to speckle transformation, or supplies the image to an image selection unit 173. The display unit 56 includes an online monitor and a microscope for viewfinder overlay.

The PC 151 performs, as offline processing, image processing of speckles on a captured image in accordance with a relationship between an image output frame rate and a sampling rate (a sampling rate equal to an image output frame rate in the case of FIG. 13).

The PC 151 includes the HDD (SSD) 171, a high-precision operation unit 172, and the image selection unit 173. The HDD 171 temporarily stores an image from the image acquisition unit 61. The high-precision operation unit 172 performs an inter-frame operation that requires inter-frame processing, as part of speckle transformation processing. The image selection unit 173 selects the image from the image output unit 63 or an image from the high-precision operation unit 172 in accordance with a control signal from the user IF 153, and causes the display unit 152 to display the selected image.

The display unit 152 includes a monitor. The user IF 153 includes a mouse, a touch panel, a keyboard, and the like, and supplies a control signal corresponding to a user operation to the image selection unit 173.

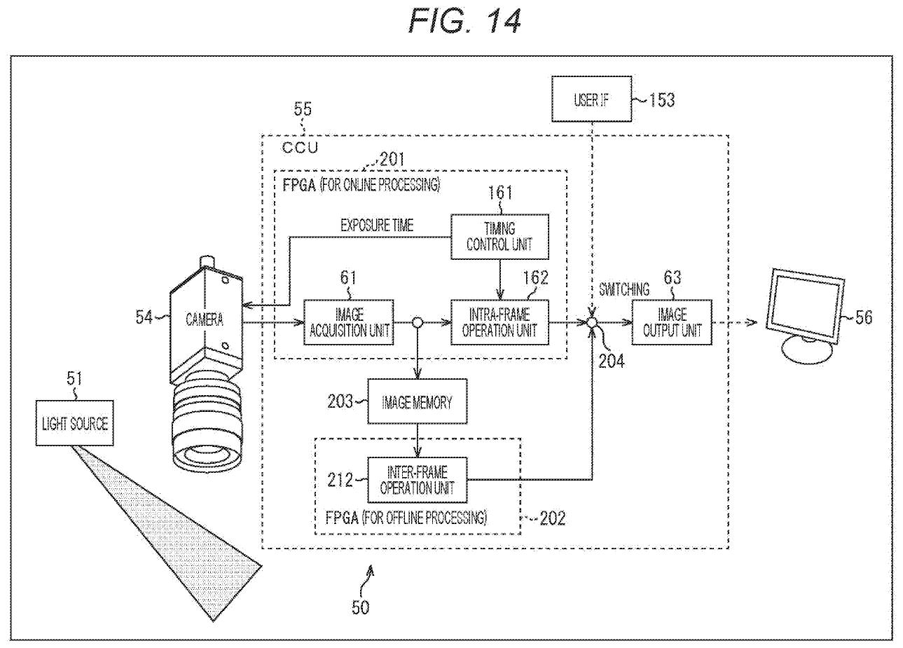

Note that the speckle imaging apparatus 50 shown in FIG. 13 is configured to perform offline processing outside the CCU 55. However, it is also possible to configure the speckle imaging apparatus 50 to perform offline processing inside the CCU 55 as shown in, for example, FIG. 14.

<Second Configuration Example of Image Processing System of Present Technology>

FIG. 14 is a block diagram showing a second configuration example of the image processing system including the speckle imaging apparatus as an image processing apparatus to which the present technology has been applied. Note that the object surface 52 and the filter 53 have been omitted, and are not shown in the example of FIG. 14.

The image processing system includes the light source 51 described above with reference to FIG. 4 and the speckle imaging apparatus 50. The speckle imaging apparatus 50 includes the camera 54, the CCU 55, the display unit 56, and the user IF 153 shown in FIG. 13.

In the example of FIG. 14, the CCU 55 includes the image output unit 63 in common with the example of FIG. 4. The CCU 55 in the example of FIG. 14 differs from that in the example of FIG. 4 in that an FPGA 201 for online processing, an FPGA 202 for offline processing, an image memory 203, and a selector 204 have been added, and the image acquisition unit 61 and the speckle transformation unit 62 have been removed. The CCU 55 performs, as online processing, image processing of speckles on a captured image in accordance with a relationship between an image output frame rate and a sampling rate (a sampling rate equal to an image output frame rate also in the case of FIG. 14).

In other words, the FPGA 201 in the example of FIG. 14 includes the image acquisition unit 61, the timing control unit 161, and the intra-frame operation unit 162 included in the CCU 55 in FIG. 13. The image acquisition unit 61 supplies an image from the camera 54 to the intra-frame operation unit 162 and the image memory 203. The intra-frame operation unit 162 outputs an image subjected to an operation to the selector 204.

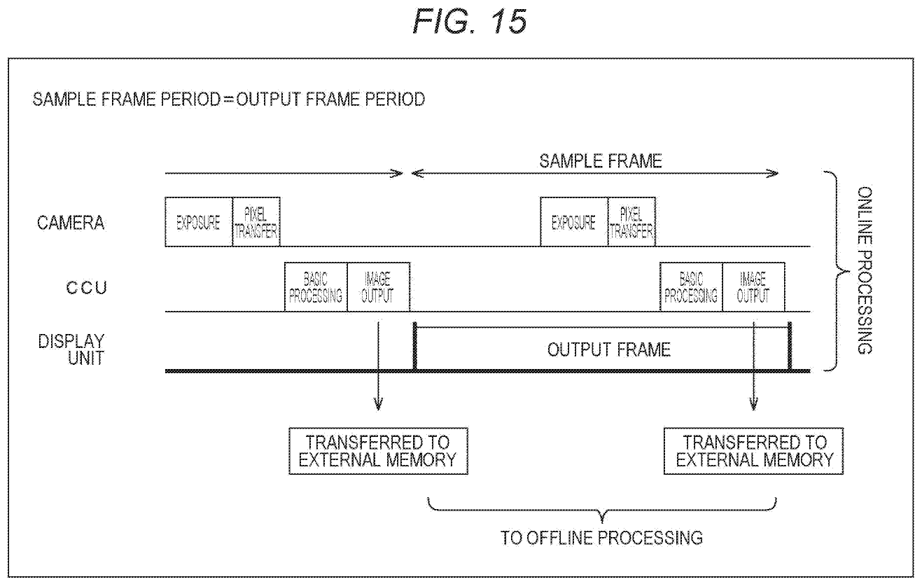

The FPGA 202 includes an inter-frame operation unit 212 that performs, as part of speckle transformation processing, an inter-frame operation on an image stored in the image memory 203. In other words, the inter-frame operation unit 212 shown in FIG. 15 performs processing that is basically similar to processing to be performed by the high-precision operation unit 172 shown in FIG. 14. The image memory 203 temporarily stores the image from the image acquisition unit 61. The inter-frame operation unit 212 performs an inter-frame operation, and supplies an image as the operation result to the selector 204.

The selector 204 selects the image from the intra-frame operation unit 162 or an image from the image memory 203 in accordance with a control signal from the user IF 153, and supplies the selected image to the image output unit 63. The user IF 153 includes a mouse, a touch panel, a keyboard, and the like, and supplies a control signal corresponding to a user operation to the selector 204.

<Operation Example of Speckle Imaging Apparatus>

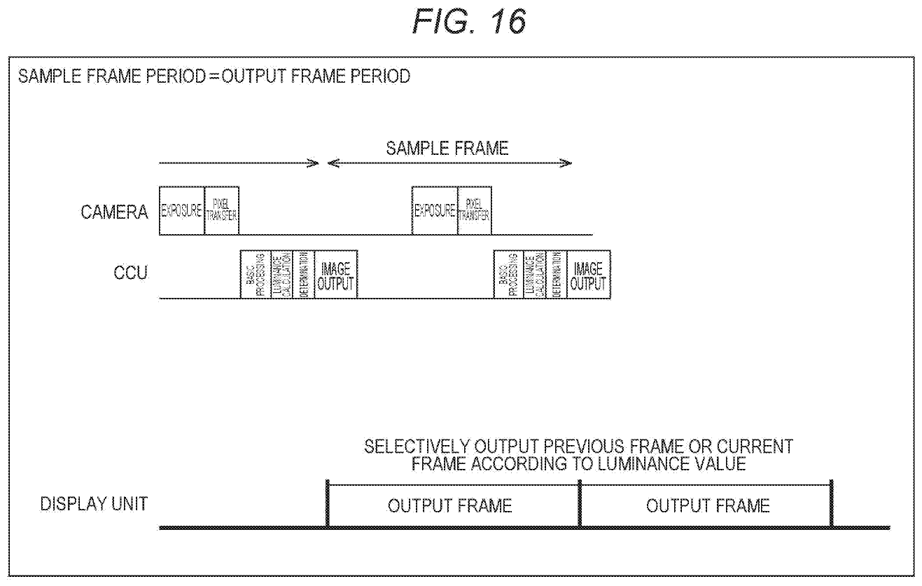

Next, an operation example of the speckle imaging apparatus shown in FIG. 13 will be described with reference to a timing chart of FIG. 15. In the speckle imaging apparatus 50 shown in FIG. 13, processing is performed such that, as shown in FIG. 16, the relationship between a sample frame period and an output frame period is expressed as follows: sample frame period=output frame period.

The camera 54 captures an image with exposure for an exposure time from the timing control unit 161, and transfers pixels of the captured image to the CCU 55. In the CCU 55, the intra-frame operation unit 162 performs basic processing via the image acquisition unit 61. Then, the image output unit 63 transfers a processed image to an external memory (for example, the HDD (SSD) 171), and causes the display unit 56 to display the processed image as an output frame. While the output frame is displayed on the display unit 56, exposure and transfer of pixels are performed by the camera 54, and the basic processing is performed in the CCU 55. Then, an image of the next frame is transferred to the external memory and displayed as an output frame on the display unit 56.

Online processing has been described above. For example, there are performed, as the basic processing, the process for distinguishability of speckles shown in FIG. 7 and the flow velocity determination process for a speckle image shown in FIG. 12, as described above.

Meanwhile, after being transferred to the external memory (for example, the HDD (SSD) 171), the image transferred to the external memory is subjected to, for example, the following offline processing as part of speckle transformation processing, performed by the high-precision operation unit 172: the above-described process for speckle vibration; the flow velocity determination process for a speckle image, shown in FIG. 12; and other inter-frame operations. Note that the above-described offline processing to be performed after the image is read from the external memory may be performed in parallel with the above-described online processing, or may be started after a certain period of time. The same applies to offline processing to be described below.

Note that the process for vibration will be described next with reference to the timing chart of FIG. 16, on the basis of an operation example in which the above-described process for vibration shown in FIG. 9 is performed online. FIG. 16 also shows an example in which processing is performed such that the relationship between a sample frame period and an output frame period is expressed as follows: sample frame period=output frame period.

The camera 54 captures an image with exposure for an exposure time from the timing control unit 161, and transfers pixels of the captured image to the CCU 55. In the CCU 55, the intra-frame operation unit 162 performs basic processing, luminance calculation, and a determination process via the image acquisition unit 61. Then, according to a result of the determination process, a processed image of the current frame or the previous frame is output by the image output unit 63 and displayed as an output frame on the display unit 56.

Note that, in the determination process, an image of the previous frame is used in a case where a calculated luminance value is equal to or more than G times the average value of the previous N frames. Meanwhile, an image of the current frame is used in a case where the calculated luminance value is less than G times the average value of the previous N frames. Here, the number N of frames as a determination criterion is optimized on the basis of vibration frequency characteristics. In addition, G for determining a determination threshold is set according to the necessity of vibration processing.

While the previous output frame is displayed on the display unit 56, exposure and transfer of pixels are performed by the camera 54, and the basic processing, the luminance calculation, and the determination process are performed in the CCU 55. In the determination process, it is determined that a calculated luminance value is equal to or more than G times the average value of the previous N frames. Then, according to the result of the determination process, an image of the previous frame is output by the image output unit 63 and displayed as an output frame on the display unit 56.

Note that the speckle imaging apparatus 50 shown in FIG. 13 has been taken as an example in describing the examples of FIGS. 15 and 16. Meanwhile, a difference between the speckle imaging apparatuses 50 shown in FIGS. 13 and 14 simply lies in whether offline processing is performed outside or inside the CCU. Thus, basically similar processing is performed, and a similar effect can be achieved also in the speckle imaging apparatus 50 shown in FIG. 14.

<Third Configuration Example of Image Processing System of Present Technology>

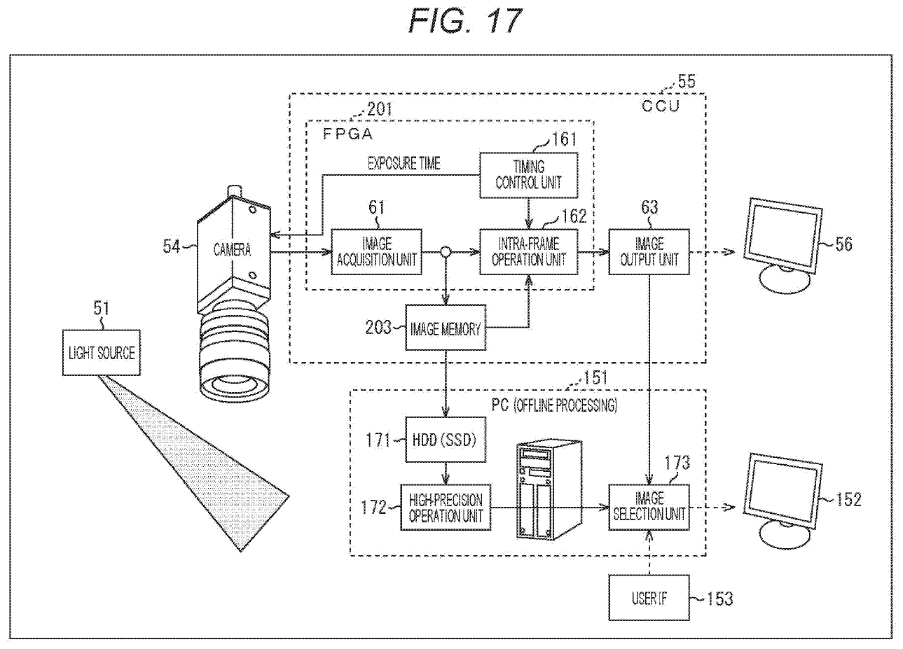

FIG. 17 is a block diagram showing a third configuration example of the image processing system including the speckle imaging apparatus as an image processing apparatus to which the present technology has been applied. Note that the object surface 52 and the filter 53 have been omitted, and are not shown in the example of FIG. 17. The speckle imaging apparatus 50 shown in FIG. 17 is an apparatus that acquires a camera image at a sampling rate higher than an image output frame rate.

The image processing system shown in FIG. 17 includes the light source 51 described above with reference to FIG. 4 and the speckle imaging apparatus 50. The speckle imaging apparatus 50 includes the PC 151, the display unit 152, and the user IF 153 shown in FIG. 13 in addition to the camera 54, the CCU 55, and the display unit 56 described above with reference to FIG. 4.

In the example of FIG. 17, the CCU 55 includes the image output unit 63 in common with the example of FIG. 4. The CCU 55 in the example of FIG. 17 differs from that in the example of FIG. 4 in that the FPRA 201 for online processing and the image memory 203 have been added, and the image acquisition unit 61 and the speckle transformation unit 62 have been removed. The CCU 55 performs, as online processing, image processing of speckles on a captured image in accordance with a relationship between an image output frame rate and a sampling rate (image output frame rate>sampling rate, in the case of FIG. 17).

In other words, the FPRA 201 in the example of FIG. 17 includes the image acquisition unit 61, the timing control unit 161, and the intra-frame operation unit 162 included in the CCU 55 in FIG. 13. The image acquisition unit 61 supplies an image from the camera 54 to the intra-frame operation unit 162 and the image memory 203. The intra-frame operation unit 162 outputs an image subjected to an operation to the image output unit 63.

As in the example of FIG. 13, the image output unit 63 causes the display unit 56 to display an image subjected to speckle transformation, or supplies the image to the image selection unit 173. The display unit 55 includes an online monitor and a microscope for viewfinder overlay.

As in the example of FIG. 13, the PC 151 is used for offline processing, and includes the HDD (SSD) 171, the high-precision operation unit 172, and the image selection unit 173.

<Operation Example of Speckle Imaging Apparatus>

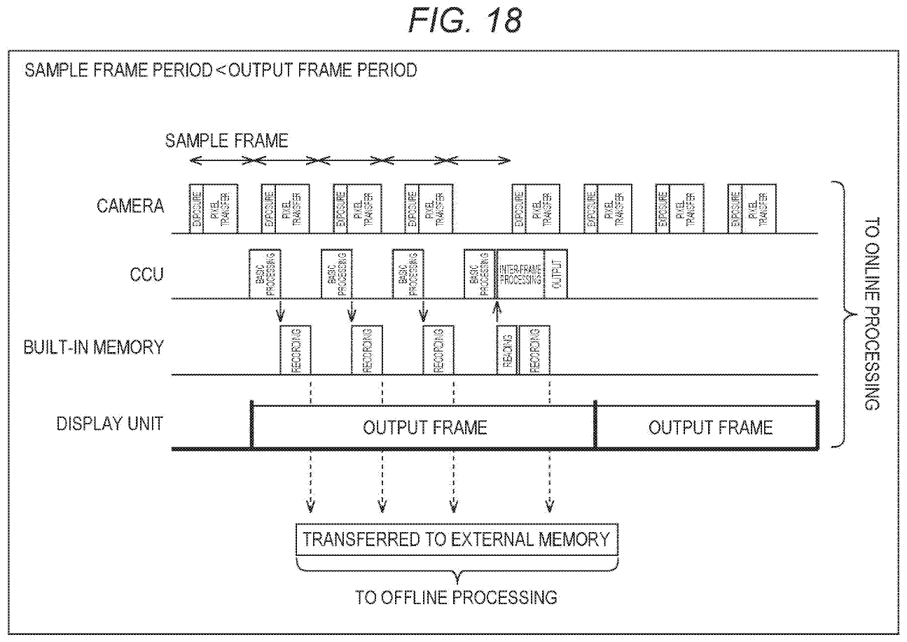

Next, an operation example of the speckle imaging apparatus shown in FIG. 17 will be described with reference to a time chart of FIG. 18. FIG. 17 shows an example in which a process for dealing with vibration is performed online as, for example, processing between a plurality of sample frame images that fits within an output frame. In the example, the above-described process is performed in the speckle imaging apparatus 50 such that, as shown in FIG. 18, the relationship between a sample frame period and an output frame period is expressed as follows: sample frame period<output frame period.

The camera 54 captures an image with exposure for an exposure time from the timing control unit 161, and transfers pixels of the captured image to the CCU 55. In the CCU 55, the intra-frame operation unit 162 performs basic processing (intra-frame processing and the like) via the image acquisition unit 61. Then, the image output unit 63 causes a processed image to be recorded in a built-in memory (for example, the image memory 203) and transferred to an external memory (for example, the HDD 171). After the above-described process including exposure, recording, and transfer (in other words, processing between four sample frame images, which fits within an output frame) is repeated four times, the CCU 55 reads an image from the built-in memory, and performs inter-frame processing on the image read from the built-in memory. Then, the image subjected to the inter-frame processing is output to be transferred to the external memory and displayed as an output frame on the display unit 56.

Exposure and transfer of pixels for the next frame are performed at the time of inter-frame processing in the CCU 55.

Meanwhile, after being transferred to the external memory (for example, the HDD (SSD) 171), the image transferred to the external memory is subjected to, for example, arithmetic processing that does not fit within an output frame. The arithmetic processing is performed offline by the high-precision operation unit 172, as part of speckle transformation processing.

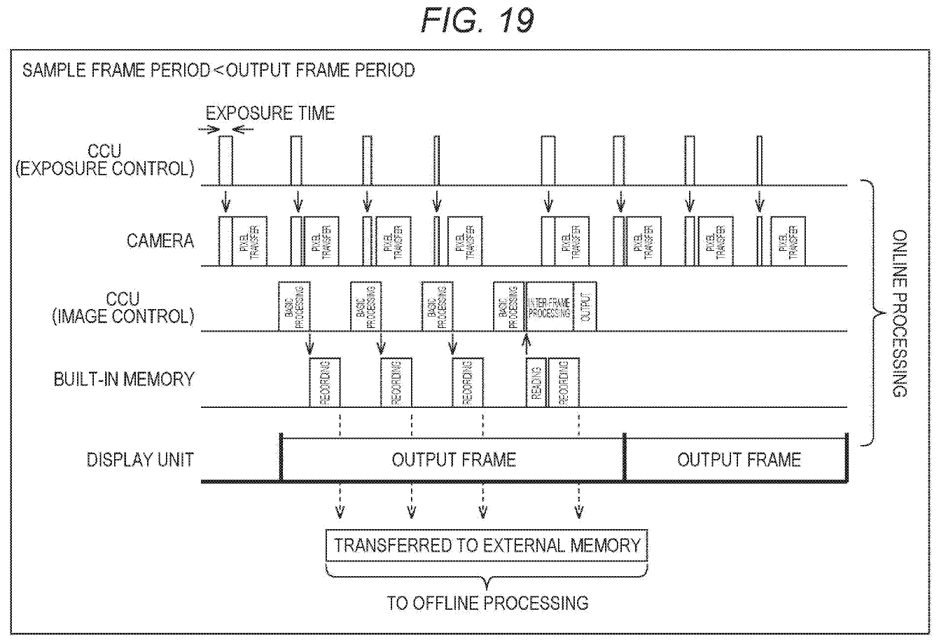

Note that in a case where the flow velocity determination process is performed online, exposure control is performed by the CCU 55 (the timing control unit 161 thereof) as shown in FIG. 19, in addition to the process of FIG. 18.

Next, the following describes an example of the inter-frame processing performed by the CCU 55, with reference to the timing charts of FIGS. 18 and 19.

For example, in a case where inter-frame processing is not performed, the average of contrasts of all frames sf01 to sf04 is used. Meanwhile, in a case where inter-frame processing is performed, for example, a frame with a different speckle contrast is excluded, and contrasts are averaged in an image. As a method for excluding a frame on such an occasion, the following methods are used: a method for calculating the overall luminance of each frame and excluding a frame having a significantly higher overall luminance than the preceding and following frames; and a method for excluding a frame with a stationary part having a contrast reduced to a value equal to or below a threshold of each frame.

Therefore, in the case where inter-frame processing is performed, contrasts are averaged as follows. Assume that it is determined that the overall luminance of, for example, the frame sf13 is significantly higher than the overall luminance of the preceding and following frames. Then, the frame sf13 is excluded, and the average of contrasts of the frames sf11, sf12, and sf14 is used thereafter.

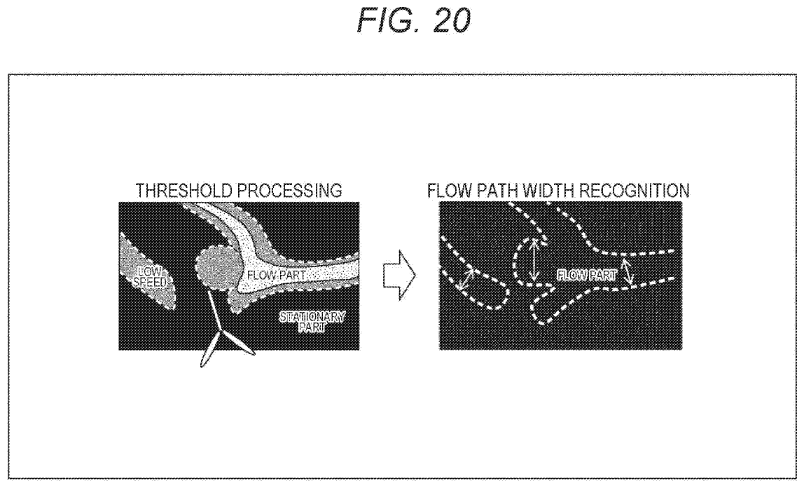

Note that threshold processing as shown in FIG. 20 may be added at the end of the basic processing. The threshold processing will be described with reference to FIG. 20.

<Threshold Processing of Speckle Image>

As shown in FIG. 20, the boundary between a flow part and a stationary part shown by a dotted line is obtained by the threshold processing to be performed at the end of the basic processing. Therefore, the width of the flow part (also referred to as a flow path) can be recognized by machine learning or the like. Note that this processing is also a kind of inter-frame processing.

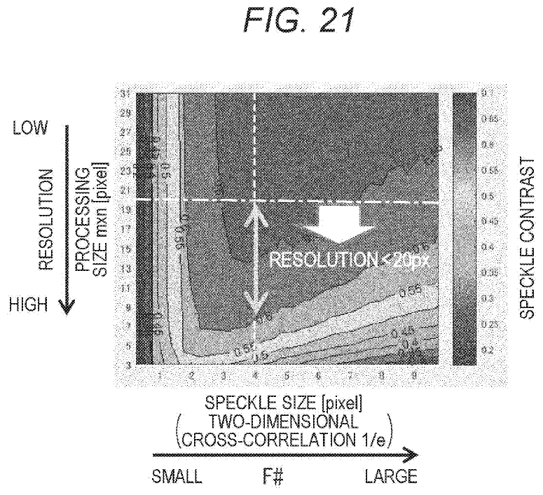

Moreover, a necessary resolution can be calculated on the basis of the width of the flow part of an object being observed. For example, assume that the width of the flow part is 100 pixels and a resolution five times the width of the flow part (→20 pixels or less) is required. The optimum processing size of speckle transformation is determined in advance from contrast characteristics determined by a speckle size and a processing size. The speckle size is determined by F# of the optical system of the speckle imaging apparatus 50. Assume that the speckle size is, for example, 4 pixels according to the specification of the optical system. Then, there is a relationship between processing size and contrast as shown on a dotted line in FIG. 21. In a case where the upper limit is set to 20 pixels corresponding to the resolution and contrast is set to 0.6 or more, a suitable processing size is 10 to 20 pixels.

As described above, according to the present technology, whether to perform image processing of speckles as online processing or offline processing on a captured image is controlled in accordance with the relationship between an image output frame rate and a sampling rate.

For example, in a case where the sampling rate is equal to the image output frame rate, image processing of speckles to be completed within a frame is performed online, and image processing of speckles that requires inter-frame processing is performed offline.

For example, in a case where the sampling rate is higher than the image output frame rate, image processing between a plurality of sample frame images in an output frame is performed online within the output frame rate, in addition to image processing of speckles to be completed within a sampling frame. Meanwhile, arithmetic processing that does not fit within the output frame rate is performed offline on a captured image stored in a memory.

As a result, both observation required to be performed on a real-time basis and highly accurate observation can be achieved at reasonable cost, so that medical quality is enhanced. Accordingly, it is possible to expect improvement in the success rate of surgery, reduction of surgical time, and reduction of medical accidents.

### 2. COMPUTER

<Computer>

A series of processes described above can be implemented by hardware, or can be implemented by software. In a case where the series of processes is implemented by software, a program included in the software is installed on a computer. Here, examples of the computer include a computer incorporated in dedicated hardware and a computer such as a general-purpose personal computer capable of performing various functions by installation of various programs.

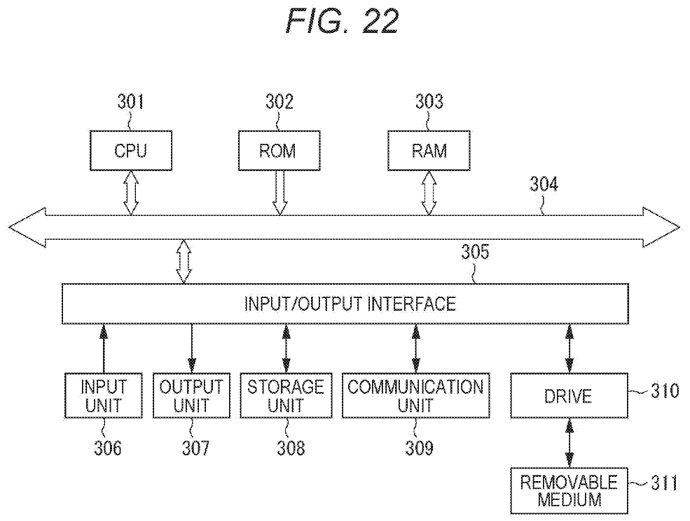

FIG. 22 is a block diagram showing a configuration example of hardware of a computer that performs the series of processes described above by means of a program.

In the computer shown in FIG. 22, a central processing unit (CPU) 301, a read only memory (ROM) 302, and a random access memory (RAM) 303 are connected to one another via a bus 304.

An input/output interface 305 is also connected to the bus 304. The input/output interface 305 is connected to an input unit 306, an output unit 307, a storage unit 308, a communication unit 309, and a drive 310.

The input unit 306 includes, for example, a keyboard, a mouse, a microphone, a touch panel, and an input terminal. The output unit 307 includes, for example, a display, a speaker, and an output terminal. The storage unit 308 includes, for example, a hard disk, a RAM disk, and a nonvolatile memory. The communication unit 309 includes, for example, a network interface. The drive 310 drives a removable medium 311 such as a magnetic disk, an optical disk, a magneto-optical disk, or a semiconductor memory.

In the computer configured as described above, a program is loaded into the RAM 303 via the CPU 301 and the bus 304 and then executed, so that the series of processes described above is performed. The RAM 303 also stores, as appropriate, data and the like necessary for the CPU 301 to perform various processes.

A program to be executed by the computer (CPU 301) can be applied after being recorded on, for example, the removable medium 311 as a package medium or the like. In this case, it is possible to mount the removable medium 311 on the drive 310 to install the program in the storage unit 308 via the input/output interface 305.

Furthermore, the program can also be provided via a wired or wireless transmission medium such as a local area network, the Internet, or digital satellite broadcasting. In this case, the program can be received by the communication unit 309 and installed in the storage unit 308.

In addition, it is also possible to install the program in the ROM 302 or the storage unit 308 in advance.

Furthermore, the embodiment of the present technology is not limited to the above-described embodiments, and various modifications may be made without departing from the gist of the present technology.

For example, in the present specification, the system refers to a set of a plurality of constituent elements (devices, modules (parts), and the like), and it does not matter whether or not all the constituent elements are in the same housing. Therefore, a plurality of devices stored in separate housings and connected via a network, and a single device including a plurality of modules stored in a single housing are both considered systems.

Furthermore, for example, the configuration described as a single device (or processing unit) may be divided and configured as a plurality of devices (or processing units). In contrast, the configurations described above as a plurality of devices (or processing units) may be integrated and configured as a single device (or processing unit). Furthermore, as a matter of course, a configuration other than that described above may be added to the configuration of each device (or each processing unit). Moreover, as long as the configuration and operation of the entire system are substantially identical, a part of the configuration of a device (or processing unit) may be included in the configuration of another device (or another processing unit).

Furthermore, in the present technology, it is possible to adopt a configuration of, for example, cloud computing in which a plurality of devices shares a single function and performs processing in collaboration with each other via a network.

Furthermore, for example, the program described above can be executed in any device. In this case, the device is only required to be configured such that the device has a necessary function (functional block or the like), and can obtain necessary information.

Furthermore, for example, each step described in the above-described flowchart can be performed by a single device, or can be shared and performed by a plurality of devices. Moreover, in a case where a plurality of processes is included in a single step, the plurality of processes included in the single step can be performed by a single device, or can be shared and performed by a plurality of devices.

Note that a program to be executed by a computer may be designed such that processes of steps in program description are performed in time series in accordance with the order in which the processes have been described in the present specification; the processes are performed in parallel; or the processes are performed separately at necessary timing at which, for example, a call is made. Moreover, the program may also be designed such that the processes of the steps in the program description are performed in parallel with processes of another program, or performed in combination with processes of another program.

Note that, as long as there is no conflict, each of a plurality of techniques of the present technology described in the present specification can be implemented independently as a single technique. As a matter of course, it is also possible to use and implement any two or more techniques of the present technology together. For example, a technique of the present technology described in any one of the embodiments can also be implemented in combination with another technique of the present technology described in another embodiment. Furthermore, any of the techniques of the present technology described above can also be used and implemented together with another technique not described above.

Note that the present technology can also adopt the following configurations.

(1) An image processing apparatus including:

an online image processing unit that performs, as online processing, image processing of speckles generated by irradiation with laser light on a captured image in accordance with a relationship between an image output frame rate and a sampling rate; and an offline image processing unit that performs, as offline processing, the image processing of speckles on the captured image.

(2) The image processing apparatus according to (1) above, in which

in a case where the captured image is acquired at a sampling rate equal to an image output frame rate, the control unit performs, as the online processing, image processing of speckles to be completed within a frame, and performs, as the offline processing, image processing of speckles that requires inter-frame processing.

(3) The image processing apparatus according to (1) above, in which

in a case where the captured image is acquired at a sampling rate equal to an image output frame rate, the control unit performs, as the online processing, image processing of speckles that requires inter-frame processing, by replacing a corresponding frame with a previous frame depending on information of a plurality of frames preceding the corresponding frame.

(4) The image processing apparatus according to any one of (1) to (3) above, in which

in a case where the captured image is acquired at a sampling rate higher than an image output frame rate, the control unit performs:

as the online processing, image processing between a plurality of sample frame images in the output frame within the output frame rate, in addition to image processing of speckles to be completed within a sampling frame; and

as the offline processing, arithmetic processing that does not fit within the output frame rate on the captured image stored in a memory.

(5) The image processing apparatus according to (4) above, in which

the control unit performs writing of the captured image to the memory and the arithmetic processing as the offline processing in parallel with the image processing of speckles as the online processing.

(6) The image processing apparatus according to (4) above, in which

the control unit performs writing of the captured image to the memory and the arithmetic processing as the offline processing after a certain period of time after the image processing of speckles as the online processing.

(7) The image processing apparatus according to any one of (1) to (6) above, in which

the inter-frame processing is processing for excluding a frame that reduces a speckle contrast of an entire image and outputting an optimum speckle contrast by complementing the excluded frame from preceding and following frames or by averaging other images in an output frame.

(8) The image processing apparatus according to any one of (1) to (7) above, in which

the inter-frame processing is processing in which:

a plurality of exposure times is set for a sample frame in the output frame rate;

a flow velocity is calculated from a contrast value for each exposure time on the basis of a previously set relational expression of a flow velocity and a contrast value for each exposure time; and

a most probable flow velocity is calculated and reflected in an image.

(9) The image processing apparatus according to any one of (1) to (8) above, in which

the inter-frame processing is processing for detecting a size of a fluid part on the basis of a different captured image and optimizing a calculation cell size so as to achieve a resolution corresponding to the detected size.

(10) The image processing apparatus according to any one of (1) to (9) above, in which

the inter-frame processing is processing including laser speckle perfusion imaging (LSPI), laser speckle flowgraphy (LSFG), or frequency domain laser speckle imaging (FDLSI) which is a calculation method using information in a time direction of speckles.

(11) The image processing apparatus according to any one of (1) to (10) above, further including:

a switching unit that causes a display image to switch between a speckle image subjected to image processing as the online processing and a speckle image subjected to image processing as the offline processing.

(12) An image processing method to be performed by an image processing apparatus, including:

controlling whether to perform image processing of speckles as online processing or offline processing on a captured image in accordance with a relationship between an image output frame rate and a sampling rate, the speckles being generated by irradiation with laser light.

(13) An image processing system including:

a light source that irradiates a surface of an object with laser light; and

an image processing apparatus including:

a control unit that controls whether to perform image processing of speckles as online processing or offline processing on a captured image in accordance with a relationship between an image output frame rate and a sampling rate, the speckles being generated by irradiation with the laser light from the light source.

### REFERENCE SIGNS LIST

10 Speckle imaging apparatus
51 Light source
53 Filter
54 Camera
55 CCU
56 Display unit
61 Image acquisition unit
62 Speckle transformation unit
63 Image output unit
71 Two-dimensional image
71-n Luminance image
72 Speckle contrast image
81 Image
82 Image
91 Image
92 Image
101 User IF
111 Untransformed image
112 Speckle operation reversed image
121 Untransformed image
122 Speckle operation reversed image
131-0 to 131-4 Input image
132-0 to 132-4 Transformed image
133-0 to 133-4 Processed image
151 Personal computer
152 Display unit
153 User IF
161 Timing control unit
162 Intra-frame operation unit
171 HDD(SSD)
172 High-precision operation unit
173 Image selection unit
201 FPGA
202 FPGA
203 Image memory
204 Selector

1. An image processing apparatus comprising:
a control unit that controls whether to perform image processing of speckles as online processing or offline processing on a captured image in accordance with a relationship between an image output frame rate and a sampling rate, the speckles being generated by irradiation with laser light.

2. The image processing apparatus according to claim 1, wherein
in a case where the captured image is acquired at a sampling rate equal to an image output frame rate, the control unit performs, as the online processing, image processing of speckles to be completed within a frame, and performs, as the offline processing, image processing of speckles that requires inter-frame processing.

3. The image processing apparatus according to claim 1, wherein
in a case where the captured image is acquired at a sampling rate equal to an image output frame rate, the control unit performs, as the online processing, image processing of speckles that requires inter-frame processing, by replacing a corresponding frame with a previous frame depending on information of a plurality of frames preceding the corresponding frame.

4. The image processing apparatus according to claim 2, wherein
in a case where the captured image is acquired at a sampling rate higher than an image output frame rate, the control unit performs:
as the online processing, image processing between a plurality of sample frame images in the output frame within the output frame rate, in addition to image processing of speckles to be completed within a sampling frame; and
as the offline processing, arithmetic processing that does not fit within the output frame rate on the captured image stored in a memory.

5. The image processing apparatus according to claim 4, wherein
the control unit performs writing of the captured image to the memory and the arithmetic processing as the offline processing in parallel with the image processing of speckles as the online processing.

6. The image processing apparatus according to claim 4, wherein
the control unit performs writing of the captured image to the memory and the arithmetic processing as the offline processing after a certain period of time after the image processing of speckles as the online processing.

7. The image processing apparatus according to claim 2, wherein
the inter-frame processing is processing for excluding a frame that reduces a speckle contrast of an entire image and outputting an optimum speckle contrast by complementing the excluded frame from preceding and following frames or by averaging other images in an output frame.

8. The image processing apparatus according to claim 2, wherein
the inter-frame processing is processing in which:
a plurality of exposure times is set for a sample frame in the output frame rate;
a flow velocity is calculated from a contrast value for each exposure time on a basis of a previously set relational expression of a flow velocity and a contrast value for each exposure time; and
a most probable flow velocity is calculated and reflected in an image.

9. The image processing apparatus according to claim 2, wherein
the inter-frame processing is processing for detecting a size of a fluid part on a basis of a different captured image and optimizing a calculation cell size so as to achieve a resolution corresponding to the detected size.

10. The image processing apparatus according to claim 2, wherein
the inter-frame processing is processing including laser speckle perfusion imaging (LSPI), laser speckle flowgraphy (LSFG), or frequency domain laser speckle imaging (FDLSI) which is a calculation method using information in a time direction of speckles.

11. The image processing apparatus according to claim 1, further comprising:
a switching unit that causes a display image to switch between a speckle image subjected to image processing as the online processing and a speckle image subjected to image processing as the offline processing.

12. An image processing method to be performed by an image processing apparatus, comprising:
controlling whether to perform image processing of speckles as online processing or offline processing on a captured image in accordance with a relationship between an image output frame rate and a sampling rate, the speckles being generated by irradiation with laser light.

13. An image processing system comprising:
a light source that irradiates a surface of an object with laser light; and
an image processing apparatus including:
a control unit that controls whether to perform image processing of speckles as online processing or offline processing on a captured image in accordance with a relationship between an image output frame rate and a sampling rate, the speckles being generated by irradiation with the laser light from the light source.
